(19) United States
(12) Patent Application Publication
(10) Pub. No.: US 2003/0143305 A1
(43) Pub. Date: Jul. 31, 2003

(54) Low fat potato chips

(76) Inventor: James Edward Maguire, Sydney (AU)

Correspondence Address: JIM MAGUIRE, 2 STRICKLAND PLACE, WENTWORTHVILLE, 2145, AU

(21) Appl. No.: 10/349,278

(22) Filed: Jan. 20, 2003

(30) Foreign Application Priority Data
Jan. 25, 2002 (AU) 13553/02

(51) Int. Cl. G01N 33/02

(52) U.S. Cl. 426/231

(57) ABSTRACT

A method for cooking low or zero fat potato chips, for hot serving and immediate consumption, which uses zero or minimal amounts of cooking oil or fat. These chips may be known as “fries” in some regions. The cooking takes place in oxygen free gas instead of cooking oil. Higher temperatures than with oil and elevated pressure are used to avoid slower heating and cooking that might otherwise follow without oil. Particular care is taken to monitor moisture content and achieve an external crispy layer which does not become soggy before the chips can be reasonably consumed, to compensate for the absence of sealing oil which slows the sogging process. A searing phase may optionally be added to thicken the crisp outer layer and further defer the onset of sogginess, to add desirable brown or bronze or golden colour, to make the low fat chips more competitive with conventional oil cooked chips.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISK APPENDIX

Not applicable.

### BACKGROUND OF THE INVENTION

1. This invention defines an improved method of cooking potato chips, without depending on oil. Subject potato chips are typically rectangular prisms 1 cm by 1 cm by 8cm, but quite variable, appearing extruded not sliced, and are served hot for immediate consumption. In some regions they may be known as “fries”.

2. Potato chips are usually cooked in oil which is used to convey heat to the chips for cooking, to exclude air and prevent burning during cooking, to add flavour which people find satisfactory, to provide some sealing of residual moisture inside the cooked chip after cooking, and to provide some lubrication to assist in chip mastication. The oil however places extra load on the digestive system and makes the end product a less healthy food. Chip outlets need also to keep a vat of near boiling oil on permanent standby.

### BRIEF SUMMARY OF THE INVENTION

3. This invention describes a method of cooking potato chips which does not depend on oil, yet produces potato chips which are crisp and palatable.

### BRIEF DESCRIPTION OF THE SEVERAL VIEWS OF THE DRAWING

Not applicable; no drawings are included.

### DETAILED DESCRIPTION OF THE INVENTION

4. In this invention, chips are cooked in hot gas, Nitrogen and Carbon Dioxide, instead of liquid, which conveys heat evenly to the chips, excludes oxygen, and removes the water given off during cooking. In one form of the invention, heated gas under pressure is introduced at the centre of a serve or typical quantity of chips in a heated cooking pan, with pneumatically sealed lid, and the gas flows steadily past the chips, collecting steam and/or water vapour, and exits the sealed cooker under pressure. The flow of gas needs firstly to displace all oxygen from the cooker, then prevent backflow of air and oxygen into the cooker while carrying away the water. In another form of the invention, the pressure inside the cooker is raised considerably above atmospheric pressure, to improve heat transfer and reduce cooking time. This can be done either at the commencement of cooking only, to more quickly bring the chips up to temperature, or throughout cooking. In each case water must still be removed during cooking. Higher concentrations of Carbon Dioxide may be used to increase gas density and improve heat transfer, as long as possible acidification of the water within the chip does not create taste problems with the potatoes used.

5. The gas has an intrinsically lower heat transfer capability than the much denser cooking oil traditionally and formerly used, which would tend to increase heating and cooking times, however with gas higher temperatures and pressures can be used, as there is no longer a limit at the temperature of boiling oil, and in the best form of the invention these higher temperatures and pressures are used to keep cooking time down to a delay acceptable to fast food and hot chip customers.

6. After oil cooking, the oil soaked crispy layer of the chip forms a temporary seal, holding in the remaining moisture. Oil cooked Potato chips can become soggy after cooking when moisture still inside the chip eventually flushes away the residual oil and penetrates the outer crisp layer. To reduce the risk of sogginess in Low Fat Potato Chips, it is best to monitor, while cooking, the steam or water vapour which the chips give off, and to continue cooking until only a small residue of water is left inside the chips, ie a controlled majority of the potato's original moisture has been driven away. Then when the chips are removed from the cooker, they will not continue to “gas” water vapour at a high rate and quickly go soggy on the outside. To best achieve this the chips should be of a uniform cross section, and a simple microprocessor or controller monitors a sensor which measures the moisture content of the exhaust gas, and gives an alarm or terminates this phase of cooking when the water flow indicates the chips are dried to the desired level. In another form of this invention a sensor measures electrical conduction between probes in a small sample of the chips during cooking and infers moisture level. In another form of this invention a sensor measures dielectric constant in a small sample of the chips during cooking and infers moisture level.

7. In another form of the invention, a “searing” phase takes place towards the end of cooking, when a required dryness is reached, to produce a thicker dry crisp layer on the outside of the chip, which layer then can provide more buffering against continued moisture outflow and sogginess. The searing is achieved with high temperature, high pressure, brief duration and little or no flow, to create a crisp outer layer penetrating to a depth of 1 or 2 mm into the chip. The searing can be accompanied by the admission of a small amount of oxygen via air, to produce a desired amount of limited browning.

8. Even though fats and oils are not needed for cooking, it is possible that, for reasons of taste and improved mastication, some customers might prefer a potato chip which does have a small quantity of oil deliberately added for these reasons, or because they find totally fat free chips too bland. This quantity can be much smaller than that which would be retained by oil cooked chips, so a health benefit would still be present, and the oil might also be used to introduce added flavours, possibly lessening the amount of oil so applied. A different variety of oil, more healthy if possible, from that commonly used for cooking, may be used. Therefore, in another form of this invention, after cooking, small quantities of possibly flavoured oil are added, possibly by spraying, to the cooked chips. This would take place in a subsequent cooking step in a separate container, so as not to contaminate the oil free cooking enclosure, which could cook chips of both types, ie zero oil and low oil, on demand.

The claims defining the invention are as follows:

1. A process of cooking potato chips which uses oxygen free gases at elevated pressure instead of cooking oil for heating the chips, and removing the water.

2. A process of cooking potato chips as in claim 1 which uses an initial burst of very high pressure to rapidly bring the chips interiors to 100 C. or higher if pressurised, then decreases the pressure for the rest of the cooking period.

3. A process of cooking potato chips as in claim 1 which uses extra high pressure, up to several atmospheres, right throughout cooking, to achieve quick cooking.

4. A process of cooking potato chips as in claim 1 which monitors the outflux of moisture from the cooking chips, over the entire period of cooking, which infers the amount of moisture remaining in the chips, and terminates or advises termination of the cooking phase when a desired remanent moisture level is reached.

5. A process of cooking potato chips as in claim 1 which measures or determines the remaining moisture level in the chips, and terminates or advises termination of the cooking phase when a desired remanent moisture level is reached.

6. A process of cooking potato chips as in claim 1 with a subsequent “searing” high pressure phase which thickens up the crisp external layer, and allows browning.

7. A process of cooking potato chips as in claim 1 which includes the subsequent addition of relatively small amounts of oil for flavouring and mastication.

8. A process of cooking potato chips as in claims 2 and 4.

9. A process of cooking potato chips as in claims 2 and 5.

10. A process of cooking potato chips as in claims 2 and 6.

11. A process of cooking potato chips as in claims 2 and 7.

12. A process of cooking potato chips as in claims 3 and 4.

13. A process of cooking potato chips as in claims 3 and 5.

14. A process of cooking potato chips as in claims 3 and 6.

15. A process of cooking potato chips as in claims 3 and 7.

16. A process of cooking potato chips as in claims 2 and 4 and 6.

17. A process of cooking potato chips as in claims 2 and 4 and 7.

18. A process of cooking potato chips as in claims 2 and 5 and 6.

19. A process of cooking potato chips as in claims 2 and 5 and 7.

20. A process of cooking potato chips as in claims 2 and 6 and 7.

21. A process of cooking potato chips as in claims 2 and 4 and 6 and 7, and in accordance with the full description herein.

22. A process of cooking potato chips as in claims 2 and 5 and 6 and 7, and in accordance with the full description herein.

23. A process of cooking potato chips as in claims 3 and 4 and 6.
